(19) United States
(12) Patent Application Publication
(10) Pub. No.: US 2026/0272949 A1
(43) Pub. Date: Sep. 17, 2026

(54) A SAFE PHARMACEUTICAL COMPOSITION OF HYDROCORTISONE

(71) Applicant: CIPLA LIMITED, Mumbai, Maharashtra (IN)

(72) Inventors: Ranjna JAIN, Kesli Sagar, Madhya Pradesh (IN); Harish SHARMA, Maharashtra (IN); Tushar RAJYAGURU, Bhavnagar, Gujarat (IN); Arunprasath KALIAPERUMAL, Weston, FL (US); Pradeep BHADAURIA, Weston, FL (US)

(21) Appl. No.: 19/473,405

(22) PCT Filed: Apr. 8, 2024
(86) PCT No.: PCT/IN2024/050364

§ 371 Date: Oct. 7, 2025

(30) Foreign Application Priority Data
Apr. 8, 2023 (IN) 202321026295

(51) Int. Cl. A61K 31/573; A61K 9/00; A61K 47/02; A61K 47/20

(52) U.S. Cl. CPC A61K 31/573; A61K 9/0019; A61K 47/02; A61K 47/20

(57) ABSTRACT

The present invention discloses an aqueous pharmaceutical composition of hydrocortisone that is free of any adverse and side effects due to the inactive ingredients used in the formulation. The present invention relates to ready to use safe stable aqueous pharmaceutical composition comprising hydrocortisone sodium phosphate comprising pH adjuster, antioxidant, buffering agent and vehicle. The present invention also discloses stability of an aqueous pharmaceutical composition comprising hydrocortisone sodium phosphate with different headspace.

### TECHNICAL FIELD

The present invention relates to an aqueous pharmaceutical composition of hydrocortisone that is free of any adverse and side effects due to the inactive ingredients used in the formulation.

### BACKGROUND ART

Glucocorticosteroids are hydrophobic molecules and are commercially available in the form of aqueous suspensions. However, the aqueous suspension of a glucocorticosteroid compound has a problem that the steroid particles precipitate as time advances. Before the administration of such compositions, it is required that the patient shakes the container before use to disperse the active component homogeneously in the liquid phase. Even in this case the particles in the suspension easily agglomerate to form cluster, thereby increasing the particle diameter of the drug and resulting in the loss of uniformity of the drug in the suspension. Such an non-uniform dispersion is not desirable and could result in improper dosing, which could in turn adversely affect the treatment of condition for which the drug is being administered.

In order to solve the problem, emulsion preparations have been proposed. Such emulsions are prepared using an oil solvent. The oil component in such formulations cause irritation, uncomfortable sensations or congestion. Thus, aqueous preparations of glucocorticosteroid without using oil solvent are desirable. Such formulations help maintain the uniformity of the drug in the solution, as well as eliminate the undesirable effects of inactive ingredients such as oil.

Hydrocortisone is one such corticosteroid used for a wide variety of uses and is known to be used as anti-inflammatory agents. It is common to use these active substances in particular for treatment of asthma, endocrine disorders, dermatological disorders and allergic states, as well as acute adrenal insufficiency.

Hydrocortisone and its salts belong to the corticosteroid family. Hydrocortisone, as well as its salts, are sensitive to oxidation. This is why the pharmaceutical solutions of these active substances always comprise at least one antioxidant. However, the antioxidants themselves used in these pharmaceutical solutions are unstable, as they reduced themselves to provide protection against oxygen. To counter the oxidation, these pharmaceutical solutions comprise an antioxidant such as disodium ethylenediaminetetraacetic acid (EDTA). EDTA stabilizes the other antioxidants used in these pharmaceutical formulations by protecting it from oxidation. For example, Efcortesol™ commercialized by Amdipharm, comprises two antioxidants-disodium EDTA and formaldehyde sodium bisulfite. Disodium EDTA serves as the chelating agent, whereas formaldehyde sodium bisulfite, which is also an antimicrobial, acts as the antioxidant in the formulation.

However, formaldehyde sodium bisulfite is not on the GRAS list (Generally Recognized As Safe) of the FDA (Food and Drug Administration). In addition, this antioxidant is not described in the international reference book referring to pharmaceutical excipients which is the Handbook of pharmaceutical excipients. Thus, it is not desirable to use formaldehyde sodium bisulfite.

U.S. Pat. No. 10,456,355 by Crossject, relate to a pharmaceutical composition of hydrocortisone comprising of two antioxidants-rongalite (also known under the name of sodium formaldehyde sulfoxylate) and disodium EDTA. The applicants state that they found that the association of these two specific antioxidants to stabilize a pharmaceutical hydrocortisone solution. However, manufacturing and commercializing of these formulations using 2 antioxidants is expensive. Thus, there is a need for a formulation that comprises a single antioxidant and is commercially more viable.

U.S. Pat. No. 2,970,944 discloses fresh aqueous solution of steroid phosphate salts are colourless and free from insoluble matter, these solutions begin to deteriorate upon standing at room temperature or at elevated temperatures to form yellow color and a precipitate. This phenomenon may be a oxidative or hydrolytic degradation. It has been disclosed that this solution was stabilized with creatinine and antioxidants like are sodium bisulfite and phenol as preservative in Water for injection.

Hydrocortone Phosphate (Hydrocortisone Sodium Phosphate) injection by Merck is an intravenous, intramuscular, and subcutaneous administration once commercialized as a single dose vial comprising sodium bisulfite as the antioxidant, creatinine as stabilizer and methylparaben and propylparaben as preservatives. This formulation is currently discontinued. Sulfite contained in this formulation may cause allergic-type reactions including anaphylactic symptoms and life-threatening or less severe asthmatic episodes in certain susceptible people. The overall prevalence of sulfite sensitivity in the general population is unknown and probably low. However, sulfite sensitivity is seen more frequently in asthmatic than in non-asthmatic people. Hydrocortisone Sodium Phosphate Injection also contains creatinine as stabilizer and may cause the renal disfunction.

Additionally, sodium bisulfite, a food additive, was banned by FDA on 8 Jul. 1986 following the deaths of 13 people and many illnesses, mainly among asthmatics. Similarly, sodium metabisulfite disinfectant, antioxidant, and preservative agent, may cause allergic reactions in those who are sensitive to sulfites, including respiratory reactions in asthmatics, anaphylaxis and other allergic reactions in sensitive individuals. Thus, such excipients are undesirable for pharmaceutical compositions intended to treat asthmaticus and acute allergic reactions, such as compositions comprising hydrocortisone sodium phosphate.

Sodium formaldehyde bisulfite, in alkaline environment slowly convert to formaldehyde and sodium bisulfite via hydrolysis and produce the desired antibacterial and antioxidant preservative properties, respectively. However, U.S. Pat. No. 4,402,943 and U.S. Pat. No. 4,555,522 disclose that certain sulfites, bisulfites, and related compounds, such as sodium formaldehyde bisulfite, display antithrombotic activity, involving anti-coagulant and/or platelet antiaggregatory activities, as well as antihypertensive activity.

The hydrocortisone formulation may cause local tissue site irritation and pain when injected subcutaneously. This is believed to be due to the high osmolality of hydrocortisone. The formulation provided herein have numerous advantages over the discontinued hydrocortisone product as the osmolality of proposed formulation is isotonic solutions (osmolality of about 300 mOsm/kg).

Based on extensive review of literature and the prior art documents and the commercially available pharmaceutical solutions, all disclosed compositions comprising hydrocortisone sodium phosphate comprise antioxidants and/or stabilizing agents which are not recommended to be used in higher concentrations in order to physiochemically stabilize and effectively preserve drug products.

A significant challenge arises to stabilize the drug product due to high oxygen sensitivity or leads to physicochemical degradation of the solution.

For parenteral drugs exclusion of oxygen from a formulation can be achieved for example by packaging the drug in glass ampoules that are heat sealed under an inert atmosphere. However, it is very difficult to fill finish the drug product which are unstable by oxidation, as the polymer stopper are reasonably permeable to oxygen.

In such cases, the criticality is to develop stable and essentially preserved drug products. For the injectable it is challenging to use excipients with least toxicity in the optimum concentrations without affecting the stability of the drug product. Furthermore, optimum concentration should provide the stability over the shelf life and should not happened that lower amount of excipients e.g antioxidant contribute in reducing the potential for toxicological effects and at the same time fail to provide sufficient protection against oxygen.

The present invention addresses the problems of the prior art knowledge by advantageously providing stable pharmaceutical composition of hydrocortisone sodium phosphate suitable for intravenous injection, which exhibit excellent shelf life after first opening (in-use shelf life).

There is a continued need for an aqueous hydrocortisone injectable that does not contain sulfite and hence is free of related side effects. It is also desirable to have a formulation of hydrocortisone that does not contain any such inactive ingredients that could have adverse effect on the patients being treated. It is also desirable to have a formulation of hydrocortisone that will be inexpensive and contain only one antioxidant and hence be commercially more viable. The present invention also includes a method of maintaining the stability of a pharmaceutical composition having Hydrocortisone sodium phosphate as the active ingredient, including the slowing down or otherwise inhibiting of the oxidation pathway of Hydrocortisone sodium phosphate by reducing the amount of oxygen in the process of manufacturing and/or storing of finished product. This can be done by replacing oxygen with nitrogen.

The instant invention concerns a pharmaceutical composition of hydrocortisone or of a pharmaceutically acceptable salt thereof to be injected by parenteral, intramuscular or subcutaneous route.

The instant invention also concerns formulation of a ready-to-use formulation of hydrocortisone. This formulation is intended to help in better compliance. It is easy to administer and need not be administered by a professional who has undergone special training for the purpose. The inventive formulation is directed at providing accurate dosing and preventing medication errors. The formulation of the current invention is provided as single dosing units to minimize loss of medication during storage and thus addresses the issue around the stability or loss of drug in the medication.

The current invention relates to such a pharmaceutical hydrocortisone composition that does not comprise sodium bisulfite. The formulation of the current invention also does not use any expensive ingredients but uses buffers that are readily available. Further, the pharmaceutical composition as per the invention, does not contain any preservative, making it commercially more viable.

### OBJECTIVE OF THE INVENTION

It is an object of the invention to develop an aqueous pharmaceutical formulation of hydrocortisone that does not contain any inactive ingredients that could have adverse effect on the patients being treated.

It is an object of the invention to develop an aqueous hydrocortisone injectable that does not contain sulfite and is free of related side effects.

It is an object of the invention to develop an aqueous hydrocortisone injectable that does not contain preservatives and to minimize the exposure to preservatives side effects.

It is an object of the invention to develop an aqueous hydrocortisone injectable that has an osmolality closer to physiological levels to minimize pain, irritation, and tissue damage.

It is also an objective to develop an aqueous pharmaceutical formulation of hydrocortisone that will be inexpensive and be commercially more viable.

According to another object of the invention the aqueous pharmaceutical formulation of hydrocortisone does not use any expensive ingredients and uses buffers that are readily available.

Another object of the invention relates to an aqueous pharmaceutical composition of hydrocortisone that does not contain any preservative, making it commercially more viable.

One objective of the invention is developing an aqueous pharmaceutical formulation of hydrocortisone that comprises monothioglycerol as an antioxidant.

Another objective of the invention is to develop an aqueous pharmaceutical formulation of hydrocortisone that comprises dibasic sodium phosphate anhydrous and monobasic sodium phosphate anhydrous as buffering agents.

It is an objective of the invention to provide a pharmaceutical composition as an aqueous pharmaceutical formulation of hydrocortisone, that is ready-to-use formulation.

It is another object of the invention to provide a pharmaceutical composition as an aqueous pharmaceutical formulation of hydrocortisone that will allow better patient compliance.

It is an objective of the invention to provide a pharmaceutical composition as an aqueous pharmaceutical formulation of hydrocortisone that need not be administered by a trained professional.

It is another objective of the invention to develop a pharmaceutical composition as an aqueous pharmaceutical formulation of hydrocortisone that allows accurate dosing and prevents medication errors during administration.

It is one more objective of the invention to develop a pharmaceutical composition as an aqueous pharmaceutical formulation of hydrocortisone that minimizes loss during storage and is stable through the process of administration.

Further objective of the invention relates to developing an aqueous pharmaceutical formulation of hydrocortisone as single dose dosing units.

One more object of present invention provides a stable, safe, efficacious, and ready to use formulation of hydrocortisone sodium phosphate or its pharmaceutically acceptable salt.

In further object is to provide processes for preparation of an aqueous pharmaceutical composition comprising hydrocortisone sodium phosphate or pharmaceutically acceptable salts thereof.

One object of the invention is to provide a stable aqueous pharmaceutical composition of hydrocortisone sodium phosphate with different concentrations of monothioglycerol including about 0.5 mg/ml, about 1.0 mg/ml and about 2.0 mg/ml monothioglycerol.

One object of the present invention relates to providing a stable composition of hydrocortisone sodium phosphate with not more than or equal to 1.0% oxygen in the headspace of a container.

Another object of the present invention relates to providing stable composition of hydrocortisone sodium phosphate with not more than or equal to 2.0% oxygen in the headspace of a container.

One another object of the present invention relates to providing stable composition of hydrocortisone sodium phosphate with not more than or equal to 4.0% oxygen in the headspace of a container.

Yet another object of the present invention relates to providing stable composition of hydrocortisone sodium phosphate with not more than or equal to 6.0% oxygen in the headspace of a container.

### SUMMARY OF INVENTION

The inventors have developed an aqueous pharmaceutical composition of hydrocortisone that is free of any adverse and side effects due to the inactive ingredients used in the formulation. The inventors have found that the formulation of the current invention accords benefits in terms of using readily available ingredients and is less expensive. The present invention more specifically relates to the following:

1. an aqueous pharmaceutical formulation of hydrocortisone that can be administered by parenteral, intramuscular, or subcutaneous route.

2. an aqueous pharmaceutical formulation of hydrocortisone that is a ready to-use formulation intended towards better patient compliance.

3. an aqueous pharmaceutical formulation of hydrocortisone that is easy to administer and does not require to be administered by a trained professional.

4. an aqueous pharmaceutical formulation of hydrocortisone that would provide accurate dosing and prevent medication errors.

5. an aqueous pharmaceutical formulation of hydrocortisone provided as single dosing units to minimize loss of medication during storage and thus address the issue around stability or loss of drug in the medication.

6. an aqueous pharmaceutical formulation of hydrocortisone that does not contain any inactive ingredients that could have adverse effect on the patients being treated.

7. an aqueous hydrocortisone injectable that does not contain sulfite and is free of related side effects.

8. an aqueous pharmaceutical formulation of hydrocortisone that will be inexpensive and be commercially more viable.

9. an aqueous pharmaceutical formulation of hydrocortisone does not use any expensive ingredients and uses buffers that are readily available.

10. an aqueous pharmaceutical composition of hydrocortisone that does not contain any preservative, making it commercially more viable.

11. an aqueous pharmaceutical formulation of hydrocortisone that comprises monothioglycerol as an antioxidant.

12. an aqueous pharmaceutical formulation of hydrocortisone that comprises dibasic sodium phosphate anhydrous and monobasic sodium phosphate anhydrous as buffering agents.

13. an aqueous pharmaceutical formulation of hydrocortisone as single dose dosing units.

The present invention further relates to a safe ready-to-use aqueous pharmaceutical composition comprising hydrocortisone sodium phosphate or its pharmaceutically acceptable salts isomers, racemates, enantiomers, hydrates, solvates, metabolites, polymorphs, and mixtures as an active agent or active ingredient. The invention particularly relates to a ready-to-use composition comprising hydrocortisone sodium phosphate or pharmaceutically acceptable salts thereof and pharmaceutically acceptable excipients.

In an embodiment of present invention, the aqueous pharmaceutical composition of hydrocortisone comprises hydrocortisone sodium phosphate in a concentration range of about 0.1% w/v to about 100% w/v.

In another embodiment of present invention, the aqueous pharmaceutical composition of hydrocortisone comprises hydrocortisone sodium phosphate in a concentration range of about 1% w/v to about 10% w/v.

In further preferred embodiment of present invention, the aqueous pharmaceutical composition of hydrocortisone comprises hydrocortisone sodium phosphate in a concentration of an amount of 6.71% w/v.

In preferred embodiment of the invention is in form of an aqueous pharmaceutical formulation of hydrocortisone that comprises dibasic sodium phosphate anhydrous, monobasic sodium phosphate anhydrous and mixture thereof as buffering agents.

In another embodiment, the aqueous pharmaceutical composition of hydrocortisone comprises a buffering agent in a concentration range of about 0.01% w/v to about 0.5% w/v, more preferably about 0.02% w/v to about 0.3% w/v.

In another embodiment, the aqueous pharmaceutical composition of hydrocortisone comprises dibasic sodium phosphate anhydrous as buffering agent in a concentration range of about 0.01% w/v to about 0.5% w/v, more preferably about 0.02% w/v to about 0.3% w/v.

In preferred embodiment of the present invention, the pharmaceutical composition comprises dibasic sodium phosphate anhydrous in an amount of about 0.22% w/v.

In another embodiment, the aqueous pharmaceutical composition of hydrocortisone comprises monobasic sodium phosphate anhydrous as buffering agent in a concentration range of about 0.02% w/v to about 0.3% w/v.

In preferred embodiment of present invention, the pharmaceutical composition comprises monobasic sodium phosphate anhydrous in an amount of about 0.04% w/v.

In an embodiment of present invention, the aqueous pharmaceutical composition of hydrocortisone comprises antioxidant in concentration range of about 0.005% w/v to about 1.0% w/v.

In one embodiment of present invention, the aqueous pharmaceutical composition of hydrocortisone comprises antioxidant in concentration range of about 0.05% w/v to about 5.0% w/v.

In preferred embodiment of the invention, the formulation comprises monothioglycerol as an antioxidant.

In an embodiment of present invention, the aqueous pharmaceutical composition of hydrocortisone comprises monothioglycerol as an antioxidant in a concentration range of about 0.01% w/v to about 0.5% w/v.

In another embodiment of present invention, the aqueous pharmaceutical composition of hydrocortisone comprises monothioglycerol as antioxidant in a concentration range of about 0.05% w/v to about 0.25% w/v.

In another preferred embodiment of present invention, the aqueous pharmaceutical composition of hydrocortisone comprises monothioglycerol in a concentration of about 0.1% w/v.

In a preferred embodiment of the present invention, the stable aqueous pharmaceutical composition of hydrocortisone sodium phosphate comprises monothioglycerol in different concentrations of about 0.1 mg/ml to about 5.0 mg/ml.

According to one preferred embodiment of the present invention the stable aqueous pharmaceutical composition of hydrocortisone sodium phosphate comprises monothioglycerol at concentrations including about 0.5 mg/ml, about 1.0 mg/ml or about 2.0 mg/ml.

In a preferred embodiment of the present invention, the composition comprises about 1.0 mg/ml of monothioglycerol.

In another embodiment, the aqueous pharmaceutical composition of hydrocortisone comprises pH adjusting agent selected from sodium borate, sodium hydroxide and sodium carbonate.

In another embodiment, the aqueous pharmaceutical composition of hydrocortisone comprises sodium hydroxide as the pH adjusting agent.

According to one embodiment, the aqueous pharmaceutical formulation of hydrocortisone has pH between of about 6 to about 9, more preferably between of about 7.5 to about 8.5.

According to one preferred embodiment, the aqueous pharmaceutical formulation of hydrocortisone has pH of about 8.0.

According to a preferred embodiment of present invention, the aqueous pharmaceutical composition of hydrocortisone comprises water for injection as vehicle.

One embodiment of the present invention relates to a safe ready-to-use aqueous pharmaceutical composition comprising: a) hydrocortisone sodium phosphate in an amount of about 67.1% w/v b) mixture of dibasic sodium phosphate in an amount of about 0.22% w/v and monobasic sodium phosphate in an amount of about 0.04% w/v and c) monothioglycerol in an amount of about 0.1% w/v.

In one embodiment, the aqueous pharmaceutical composition according to present invention can be prepared by process comprising following steps:

a) collecting the sufficient quantity of required vehicle in the manufacturing vessel and flushing inert gas into it till the dissolved oxygen content is achieved.

b) maintaining an inert gas condition and suitably adding antioxidant, buffering agent into step a), stirring the solution till complete dissolution.

c) Adding active ingredients into step b) by maintaining the suitable conditions till complete dissolution.

d) Adjusting the pH of the solution with the help of pH adjuster.

e) Making up the volume with the vehicle.

f) filtering it and filling it into sterilized containers.

In one preferred embodiment, the aqueous pharmaceutical composition according to present invention can be prepared by process comprising the following steps:

a) Collecting water for injection in jacketed SS compounding/manufacturing vessel and purging the nitrogen till the dissolved oxygen content is achieved.

b) Adding monothioglycerol, dibasic sodium phosphate anhydrous, monobasic sodium phosphate anhydrous and hydrocortisone sodium phosphate USP, under continuous stirring

c) Adding hydrocortisone sodium phosphate USP acid under continuous stirring and nitrogen purging in step (b) and stirring the solution till complete dissolution.

d) Checking and adjusting the pH of solution to 8.0

e) Making up the volume with water for injection to 100% of the batch volume and stirring

f) filtering the solution of step (e) and filling into sterilized container.

The stable pharmaceutical composition according to one embodiment uses not more than or equal to about 1.0% oxygen to about not more than or equal to about 6.0% oxygen by volume of the headspace in the container.

In preferred embodiment of the present invention, the stable pharmaceutical composition uses not more than or equal to 1.0% oxygen in headspace container.

A preferred embodiment of the present invention relates to the aqueous pharmaceutical composition of hydrocortisone sodium phosphate with not more than or equal to 1.0% oxygen in the headspace of a container.

One preferred embodiment of the present invention relates to an aqueous pharmaceutical composition of hydrocortisone sodium phosphate with not more than or equal to 2.0% oxygen in the headspace of a container.

Another preferred embodiment of the present invention relates to an aqueous pharmaceutical composition of hydrocortisone sodium phosphate with not more than or equal to 4.0% oxygen in the headspace of a container.

In a preferred embodiment of the present invention the aqueous pharmaceutical composition of hydrocortisone sodium phosphate uses not more than or equal to 6.0% oxygen in the headspace of a container.

In preferred embodiment of the present invention the aqueous pharmaceutical composition of hydrocortisone sodium phosphate remains stable for at least 12 months, preferably at least 9 months or more preferably, at least 6 months and furthermore preferably for 3 months at 25° C./60% RH (relative humidity) with low oxygen levels of not more than or equal to 1.0% oxygen in the headspace of a container.

In another preferred embodiment of the invention, the composition remains stable for at least 12 months at 25° C./60% RH (relative humidity) with low oxygen levels of not more than or equal to 1.0% oxygen in the headspace of a container.

In another preferred embodiment of present invention, the aqueous pharmaceutical composition is stable with lower oxygen levels in the headspace, correlated with the lower color value developed during storage.

In preferred embodiment of present invention, the aqueous pharmaceutical composition does not contain sulfite.

In preferred embodiment of present invention, the aqueous pharmaceutical composition is administered by parenteral, intramuscular, or subcutaneous route.

### DETAILED DESCRIPTION

Hydrocortisone or cortisol is a glucocorticoid secreted from the adrenal gland and equipped with anti-inflammatory activity capable of releasing and inducing the synthesis of the specific PLA2 inhibitor (lipocortin) therefore blocking the arachidonate cascade and the formation of phlogogenic factors, like prostaglandins, thromboxanes, (SRS-A) leukotrienes. Such a mechanism of action explains the anti-inflammatory and anti-allergic activity of hydrocortisone.

The chemical name for Hydrocortisone sodium phosphate is. Disodium; [2[(8S,9S,10R,11S,13S,14S,17R)-11,17-dihydroxy-10,13-dimethyl-3-oxo 2,6,7,8,9,11,12,14,15,16-decahydro-1H-cyclopenta[a]phenanthren-17-yl]-2-oxoethyl]phosphate. The structural formula is

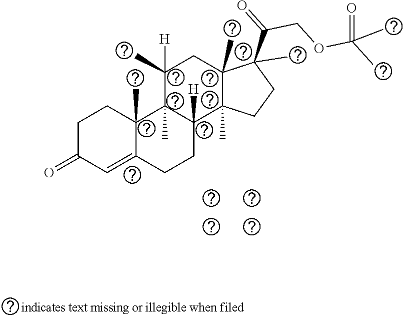

As used in the present specification, the following words, phrases, and symbols are generally intended to have the meanings as set forth below, except to the extent that the context in which they are used indicates otherwise.

As used herein, the term “about” may be used to specify a value of a quantity or parameter (e.g., the length of an element) to within a continuous range of values in the neighborhood of (and including) a given (stated) value. For example, “about 100” means any value between 90 and 110, including 90 and 110 such as 90, 91, 92, 93, 94, 95, 96, 97, 98, 99, 100, 101, 102, 103, 104, 105, 106, 107, 108, 109, and/or 110 and fractions in-between.

The term used herein, “not more than (NMT)” refers to specifying that the variable or quantity must be less or equal to the specified limit. For example, not more than five could be 1, 2, 3, 4, or 5.

The term “not less than (NLT)” indicates that the value is greater than the given value and the given value is also included. For example, not less than five could be 5, 6, 7, 8, 9,10 and higher.

As used herein and unless otherwise stated, the term “concentration” refers to the weight percent concentration. Also, the singular forms “a”, “an” and “the” include plural references unless the context clearly dictates otherwise.

The term “composition” or “formulation” or “dosage form” has been employed interchangeably for the purpose of the present invention and means that it is a pharmaceutical composition which is suitable for administration to a patient or subject. The subject can be an animal, preferably a mammal, more preferably a human.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

As used herein, the word “injectable”, or phrase “suitable for injection” refers to a composition that can be injected through a hypodermic needle safely into human or animal patients when (1) each of the components in the composition has been used in a marketed injection drug approved by the FDA at the time of this application, (2) the composition can be expelled manually using a syringe through a needle smaller than 21 gauge, preferably 25 gauge and more preferably 27 gauge, (3) the composition is free of any particles of size >10 micron in diameter, and (4) the osmolality of the composition is less than 1000 mOsmol/Kg, preferably less than 500 mOsmol/Kg centipoises and more preferably less than 380 mOsmol/Kg.

### Pharmaceutical Compositions

The present invention relates to safe, ready-to-use aqueous pharmaceutical composition comprising hydrocortisone sodium phosphate or its pharmaceutically acceptable salts isomers, racemates, enantiomers, hydrates, solvates, metabolites, polymorphs, and mixtures thereof. The invention particularly relates to a ready-to-use composition comprising hydrocortisone sodium phosphate or pharmaceutically acceptable salts thereof and pharmaceutically acceptable excipients.

The term “active ingredient” or active agent” are used interchangeably. These terms signify any ingredient that provides biologically active or other direct effect in the diagnosis, cure, mitigation, treatment, or prevention of disease or to affect the structure or any function of the body of humans or animals.

The term “pharmaceutically acceptable excipient” denotes any of the components of pharmaceutical formulation other than the active agent. A combination of excipient may also be used. The amount of excipient(s) employed may depend upon how much active agent is to be used. One excipient may perform more than one function.

The term “safe” generally refers to the formulation according to the invention which is free of adverse effects on the patient. It could also mean a formulation according to the invention that results in minimal pain, irritation and related side effects.

The term “ready-to use” or “RTU” when used in conjunction with medications presented herein refers to providing high quality intravenous preparations while simplifying the process of delivering small volume parenteral without prior need for dilution or other adjustment such as addition of saline or other tonicity agents. The RTU system facilitates efficiency, accuracy, and safety.

In an embodiment of present invention, the aqueous pharmaceutical composition is in form of ready-to-use formulation intended towards better patient compliance.

The present invention relates to an aqueous pharmaceutical composition of hydrocortisone that is free of any adverse and side effects due to the inactive ingredients used in the formulation. The instant invention provides an aqueous pharmaceutical formulation of hydrocortisone that is patient compliant and does not need a skilled professional to administer the same. The formulation provides for accurate dosing and is devoid of any medication errors.

The inventive formulation of the current invention accords benefits in terms of using readily available ingredients and is less expensive. The formulation of the current invention can be administered by parenteral, intramuscular, or subcutaneous route. The present invention provides an aqueous pharmaceutical formulation of hydrocortisone that is a ready-to-use formulation and is devoid of any issues around stability during storage.

In an embodiment of an aqueous pharmaceutical composition of present invention comprising hydrocortisone sodium phosphate or pharmaceutical acceptable salts thereof and pharmaceutically acceptable excipient.

In an embodiment of present invention, the aqueous pharmaceutical composition of hydrocortisone comprises hydrocortisone sodium phosphate selected as active ingredients in a concentration range of about 0.1% w/v to about 100% w/v.

In another embodiment of present invention, the aqueous pharmaceutical composition of hydrocortisone comprises hydrocortisone sodium phosphate selected as active ingredients in a concentration range of about 1% w/v to about 10% w/v.

In further preferred embodiment of present invention, the aqueous pharmaceutical composition of hydrocortisone comprises hydrocortisone sodium phosphate in a concentration of an amount of 6.71% w/v.

In an embodiment of present invention, the aqueous pharmaceutical composition of hydrocortisone comprises hydrocortisone sodium phosphate selected as active ingredients in a concentration range of about 2 mg/2 ml to about 2000 mg/2 ml or, about 1 mg/ml to about 1000 mg/ml.

In another embodiment of present invention, the aqueous pharmaceutical composition of hydrocortisone comprises hydrocortisone sodium phosphate selected as active ingredients in a concentration range of about 20 mg/2 ml to about 200 mg/2 ml; or about 10 mg/ml to about 100 mg/ml.

In preferred embodiment of present invention, the aqueous pharmaceutical composition of hydrocortisone comprises hydrocortisone sodium phosphate in a concentration of an amount of 134.2 mg/2 ml.

In another preferred embodiment of present invention, the aqueous pharmaceutical composition of hydrocortisone comprises hydrocortisone sodium phosphate selected as active ingredients in a concentration of an amount of 67.1 mg/ml.

The pharmaceutical compositions of the current invention do not contain preservatives, sulfite and are free of related side effects. The compositions comprise readily available inactive ingredients.

The pharmaceutical composition of the current invention may be prepared using the glucocorticosteroid compound that may be formulated as a composition further comprising a dispersant, stabilizer, antioxidant, buffering agent, pH adjuster, a carrier and/or a preservative.

In an embodiment of present invention, the aqueous pharmaceutical composition of hydrocortisone comprises antioxidant, buffering agent, pH adjuster and vehicle.

The pharmaceutical composition can be formulated as a parenteral administration form including injection for intravenous administration, intramuscular administration or subcutaneous administration, intravenous drip, transdermal absorber, transmucosal absorber, eye drop, ear drop, nose drop or inhaler. The aqueous suspension or pharmaceutical composition can be in the form of a kit, accompanying an outer package, a container, and/or an instruction for preparation/administration. When the aqueous suspension or pharmaceutical composition is provided in the form of a kit, different components may be individually packed in separate containers and contained in a single kit. Alternatively, more than one but not all of the components may be included in the kit, and other components may be provided separately from the kit.

The present invention includes a kit and/or pharmaceutical container for holding the hydrocortisone sodium phosphate compositions described herein. In accordance with current FDA requirements, vials containing the inventive formulations contain below the acceptable limits for particulate matter. Thus, the vials contain:
Particles=10 μm: Not more than 6000 per container (average)
Particles=25 μm: Not more than 600 per container (average).

The compositions of the present invention can be packaged in any suitable sterile vial or container fit for the sterile storage of a pharmaceutical such as hydrocortisone sodium phosphate for extended periods of time. Suitable containers can be glass vials, i.e. Schott treated vials, molded glass vials and CZ resin vials, polypropylene or polyethylene vials or other special purpose containers. Preferably molded glass vials.

Compositions of the invention can be supplied in unit dose vials or ampoules, having the headspace filled by an inert gas. Preferably, the containers are protected from light and stored at temperatures from about 15° C. to about 30° C.

In another embodiment, an aqueous pharmaceutical composition comprising hydrocortisone sodium phosphate or its pharmaceutically acceptable salts thereof wherein the composition is terminally sterilized and packed in unit dose glass vial. The pharmaceutical composition of the invention can be suitably produced by a known method.

The dose and number of administration of the pharmaceutical composition are not limited and can be suitably selected at a physician's discretion depending on purpose of prevention of deterioration/progress and/or the purpose of treatment of the disease to be treated, the type of disease, and patient's conditions such as body weight and age. The dose is generally about 0.01 to 1000 mg (on an active component weight basis) a day for an adult and can be administered once or in several times a day. The administration can be carried out once, twice, or numerous times, weekly, biweekly, fortnightly, once in a quarter or once in 6 months, or as required by the dosing regimen. The administration may be continued till the treatment is completed. The administration route is an injection or topical administration, for example, intravenous injections, intramuscular injections or subcutaneous injections, intravenous drips, eye drops, ear drops, nose drops, transdermal administration, transmucosal administration or inhalation.

In an embodiment of present invention, the aqueous pharmaceutical formulation of hydrocortisone is provided as single dosing units to minimize loss of medication during storage and thus address the issue around stability or loss of drug in the medication. Additionally, since the formulation provided herein may use multidose vial, it provides greater flexibility at the pharmacy and clinics for healthcare providers to prepare different dose combinations for multiple patients.

In preferred embodiment of present invention, the aqueous pharmaceutical composition of hydrocortisone administered as single dose dosing unit.

The term “buffer” or “buffering agent” are used interchangeably in the present invention. Buffering agents are useful in the present invention for, among other purposes, manipulation of the total pH of the pharmaceutical formulation. A variety of buffers known in the art may be used in the present formulations, such as various salts of organic or inorganic acids, bases, or amino acids, and including various forms of citrate, phosphate, tartrate, succinate, adipate, maleate, lactate, acetate, bicarbonate, or carbonate ions or combinations thereof. In an embodiment of the present invention, the aqueous pharmaceutical composition of hydrocortisone comprises one or more buffering agents. One preferred embodiment of the invention is in form of an aqueous pharmaceutical formulation of hydrocortisone that comprises dibasic sodium phosphate anhydrous, monobasic sodium phosphate anhydrous or mixture thereof as buffering agents.

In another embodiment, the aqueous pharmaceutical composition of hydrocortisone comprises a buffering agent in a concentration range of about 0.01% w/v to about 0.5% w/v, more preferably about 0.02% w/v to about 0.3% w/v.

In another embodiment, the aqueous pharmaceutical composition of hydrocortisone comprises dibasic sodium phosphate anhydrous as buffering agent in a concentration range of about 0.01% w/v to about 0.5% w/v, more preferably about 0.02% w/v to about 0.3% w/v.

In preferred embodiment of present invention, the pharmaceutical composition comprising dibasic sodium phosphate anhydrous in an amount of about 0.22% w/v. In another embodiment, the aqueous pharmaceutical composition of hydrocortisone comprises monobasic sodium phosphate anhydrous as buffering agent in concentration range of about 0.02% w/v to about 0.3% w/v.

In preferred embodiment of present invention, the pharmaceutical composition comprises monobasic sodium phosphate anhydrous in an amount of about 0.04% w/v.

In another embodiment, the aqueous pharmaceutical composition of hydrocortisone comprises the buffering agent in a concentration range of about 0.2 mg/2 ml to about 10 mg/2 ml or, about 0.1 mg/mL to about 5.0 mg/ml, preferably about 0.4 mg/2 ml to about 6.0 mg/2 ml, or more preferably 0.2 mg/mL to about 3.0 mg/ml.

In another embodiment, the aqueous pharmaceutical composition of hydrocortisone comprises dibasic sodium phosphate anhydrous as buffering agent in concentration range of about 0.1 mg/ml to about 5.0 mg/ml, more preferably 0.2 mg/mL to about 3.0 mg/ml.

In a preferred embodiment of present invention, the pharmaceutical composition comprises dibasic sodium phosphate anhydrous in an amount of about 2.2 mg/ml. In another embodiment, the aqueous pharmaceutical composition of hydrocortisone comprises monobasic sodium phosphate anhydrous as buffering agent in concentration range of about 0.2 mg/ml to about 1 mg/ml.

In a preferred embodiment of present invention, the pharmaceutical composition comprises monobasic sodium phosphate anhydrous in an amount of about 0.4 mg/ml.

In another embodiment, the aqueous pharmaceutical composition of hydrocortisone comprises dibasic sodium phosphate anhydrous as buffering agent in a concentration range of about 0.2 mg/2 mL to about 10 mg/2 mL, more preferably about 0.4 mg/2 ml to about 6.0 mg/2 ml.

In a preferred embodiment of present invention, the pharmaceutical composition comprises dibasic sodium phosphate anhydrous in an amount of about 4.4 mg/2 ml. In another embodiment, the aqueous pharmaceutical composition of hydrocortisone comprises monobasic sodium phosphate anhydrous as buffering agent in concentration range of about 0.4 mg/2 ml to about 2 mg/2 ml.

In preferred embodiment of present invention, the pharmaceutical composition comprises monobasic sodium phosphate anhydrous in an amount of about 0.8 mg/2 mL.

In another embodiment, the aqueous pharmaceutical composition of hydrocortisone comprises a buffering agent in concentration range of about 1 mmol to about 50 mmol, more preferably about 1 mmol to about 20 mmol.

In another embodiment, the aqueous pharmaceutical composition of hydrocortisone comprises dibasic sodium phosphate anhydrous as a buffering agent in concentration range of about 10 mmol to about 20 mmol.

Yet in another preferred embodiment, the pharmaceutical composition of the present invention, dibasic sodium phosphate anhydrous in concentration of about 15.49 mmol is used as the buffering agent.

In another embodiment, the aqueous pharmaceutical composition of hydrocortisone comprises monobasic sodium phosphate anhydrous as buffering agent in a concentration range of about 1 mmol to about 10 mmol.

In a preferred embodiment, the pharmaceutical composition of the present invention comprises monobasic sodium phosphate anhydrous in a concentration of about 3.33 mmol.

Hydrocortisone, as well as its salts, are sensitive to oxidation. This is why the pharmaceutical composition of these active substances always comprise at least one antioxidant. However, the antioxidants themselves used in this pharmaceutical composition are unstable, as they reduced themselves to provide protection against oxygen. To counter the oxidation, the pharmaceutical composition of present invention further comprises antioxidant. Suitable antioxidants include those, although not limited to, monothioglycerol, L-cysteine and thioglycolic acid, selected from the group comprising of acetyl cysteine, butylated hydroxy toluene, butylated hydroxy anisole, DL-tocopherol, sodium metabisulfite, sodium formaldehyde sulfoxylate, EDTA and its derivatives, methionine, ascorbic acid, citric acid, and its pharmaceutically acceptable salt. Antioxidants used in the formulation of the current invention have superior oxygen scavenging ability and can be easily used to scavenge oxygen smoothly. According to one preferred embodiment of the invention, an aqueous pharmaceutical formulation of hydrocortisone comprises at least one antioxidant. According to another preferred embodiment of the invention, the formulation comprises monothioglycerol as an antioxidant.

In an embodiment of present invention, the aqueous pharmaceutical composition of hydrocortisone comprises antioxidant in concentration range of about 0.1 mg/2 ml to about 20.0 mg/2 ml, or about 0.05 mg/ml to about 10.0 mg/ml.

In another embodiment of present invention, the aqueous pharmaceutical composition of hydrocortisone comprises antioxidant in concentration range of about 1 mg/2 ml to about 10 mg/2 ml, or about 0.5 mg/ml to about 5.0 mg/ml.

In an embodiment of present invention, the aqueous pharmaceutical composition of hydrocortisone comprises monothioglycerol as antioxidant in concentration range of about 0.2 mg/2 ml to about 10.0 mg/2 ml, or about 0.1 mg/ml to about 5.0 mg/ml. In another embodiment of present invention, the aqueous pharmaceutical composition of hydrocortisone comprises monothioglycerol as antioxidant in a concentration range of about 1.0 mg/2 ml to about 5.0 mg/2 ml., or about 0.5 mg/ml to about 2.5 mg/ml.

In preferred embodiment of present invention, the aqueous pharmaceutical composition of hydrocortisone comprises monothioglycerol in a concentration of about 1.0 mg/ml.

According to yet another preferred embodiment of present invention, the aqueous pharmaceutical composition of hydrocortisone comprises monothioglycerol in concentration of about 2.0 mg/2 ml.

In an embodiment of present invention, the aqueous pharmaceutical composition of hydrocortisone comprises antioxidant in concentration range of about 0.005% w/v to about 1.0% w/v.

In one embodiment of present invention, the aqueous pharmaceutical composition of hydrocortisone comprises antioxidant in concentration range of about 0.05% w/v to about 5.0% w/v.

In an embodiment of present invention, the aqueous pharmaceutical composition of hydrocortisone comprises monothioglycerol as antioxidant in concentration range of about 0.01% w/v to about 0.5% w/v.

In another embodiment of present invention, the aqueous pharmaceutical composition of hydrocortisone comprises monothioglycerol as antioxidant in concentration range of about 0.05% w/v to about 0.25% w/v.

In a preferred embodiment of present invention, the aqueous pharmaceutical composition of hydrocortisone comprises monothioglycerol concentration of about 0.1% w/v.

In an embodiment of present invention, the aqueous pharmaceutical composition of hydrocortisone comprises antioxidant in concentration range of about 0.1 mmol to about 50.0 mmol.

In further embodiment of present invention, the aqueous pharmaceutical composition of hydrocortisone comprises monothioglycerol in concentration range of about 1 mmol to about 20.0 mmol.

In another preferred embodiment of present invention, the aqueous pharmaceutical composition of hydrocortisone comprises monothioglycerol concentration of about 9.24 mmol.

The instant invention provides according to one preferred embodiment, an aqueous pharmaceutical formulation of hydrocortisone that comprises pH adjusters to adjust the pH between of about 6 to about 9, more preferably between of about 7.5 to about 8.5 and most preferably to about 8.0. According to one embodiment of the invention, any of the known pH adjusters may be used to adjust the pH to the said range. According to a preferred embodiment, sodium hydroxide may be used as a pH adjuster to adjust the pH. Any acid such as hydrochloric acid may be used to adjust the pH according to another preferred embodiment of the current invention. The formulation of present invention further includes a pharmaceutically acceptable vehicle which is suitable for pharmaceutical use such as water for injection, dextrose solution, lactate solution, saline, D5W etc. In preferred embodiment of present invention, the aqueous pharmaceutical composition of hydrocortisone comprises water for injection as vehicle.

According to an aspect of present invention, there is provided stable, safe, efficacious and ready to use formulation of hydrocortisone sodium phosphate or its pharmaceutically acceptable salt. Also disclosed herein are processes for preparation of an aqueous pharmaceutical composition comprising hydrocortisone sodium phosphate or its pharmaceutically acceptable salts thereof.

In one embodiment, the aqueous pharmaceutical composition according to present invention can be prepared by process comprising following steps:

a) collecting the sufficient quantity of required vehicle in the manufacturing vessel and flushing inert gas into it till the dissolved oxygen content is achieved.

b) maintaining an inert gas condition and suitably adding antioxidant, buffering agent into step a), stirring the solution till complete dissolution.

c) Adding active ingredients into step b) by maintaining the suitable condition till complete dissolution.

d) Adjusting the pH of the solution with the help of pH adjuster, if necessary.

e) Making up the volume with the vehicle and stirring the solution.

f) filtering it and filling it into terminally sterilized containers by maintaining an inert gas condition.

In one preferred embodiment, the aqueous pharmaceutical composition according to present invention can be prepared by process comprising following steps:

a) Collecting water for injection in jacketed SS compounding/manufacturing vessel and purging the nitrogen till the dissolved oxygen content is achieved.

b) Adding monothioglycerol, dibasic sodium phosphate anhydrous, monobasic sodium phosphate anhydrous and hydrocortisone sodium phosphate USP, under continuous stirring and nitrogen purging in step (a) and stirring the solution till complete dissolution.

c) Adding hydrocortisone sodium phosphate USP acid under continuous stirring and nitrogen purging in step (b) and stirring the solution till complete dissolution.

d) Checking and adjusting the pH of solution to 8.0.

e) Making up volume with water for injection to 100% of the batch volume

f) filtering the solution of step (e) and filling into sterilized container.

The term “stable” refers to an injection composition of present invention which is physically as well as chemically stable as demonstrated by compliance to acceptable specification when the composition is stored at convenient temperature, such as between about 0° C. and about 60° C., for a commercially reasonable period of time, such as at least about 1 day, at least about 1 week, at least about 1 month, at least about 3 months, at least about 6 months, at least about 1 year, or at least about 2 years.

The term “stable” also refers to an aqueous composition as per the present invention which exhibits physical stability for a sufficiently long time to allow for infusion of the product. The injectable suitably, the pharmaceutical composition of hydrocortisone sodium phosphate of present invention remains physically stable, with no precipitation or crystallization or color change upon storage and the shelf life period of 18-24 months when stored at 25° C. with 60% RH wherein various parameters such as the assay and degradation of related substances, i.e. known and unknown impurities remains within specified limits such as those specified according to ICH guidelines, upon storage for prolonged period of time such as for at least 12 months, preferably for 18 months, more preferably 24 months or longer. The formulation of present invention exhibits acceptable stability, retains a pharmaceutically desirable appearance, maintains the desired stability. Further, the formulation provided herein is suitable for parental dosage.

While not wishing to be bound by any theory whatsoever, it is believed that the use of anhydrous excipients such as, buffering agent, antioxidants, pH adjuster in preparing hydrocortisone sodium phosphate compositions of the invention play a significant role in reducing the degradation of hydrocortisone thereby prolonging the shelf-life of said hydrocortisone compositions.

An aqueous pharmaceutical composition of the present invention also exhibits sufficient stability. The inventors of the present invention through rigorous experimentation have found out that headspace oxygen process and concentration of antioxidant imparts sufficient stability to the injectable composition of hydrocortisone sodium phosphate or its pharmaceutically acceptable salts thereof. As stated hereinabove, the oxygen scavenger monothioglycerol in composition of this invention has a very good ability to scavenge oxygen and is suitably used to provide an atmosphere which is free of oxygen or contains a markedly reduced amount of oxygen. The water for injection in these compositions greatly affects the oxygen scavenging property of the monothioglycerol. Therefore, according to one embodiment of the present invention of the stable aqueous pharmaceutical composition of hydrocortisone sodium phosphate uses monothioglycerol in a concentration of about 0.5 mg/ml, about 1.0 mg/ml and about 2.0 mg/ml.

In preferred embodiment of present invention, the composition with about 1.0 mg/ml of monothioglycerol was selected for further process.

As used herein the term “headspace” refers to an unfilled area in a container carrying drug composition. The container is typically sealed to prevent exchange of any gasses present above the filled composition portion of the container with the external gaseous environment outside of the sealed container.

The meticulous regulation of oxygen levels within the headspace of the composition yields advantageous outcomes in reducing the aggregate oxygen content across the entire composition, encompassing both the container headspace and the dissolved oxygen absorbed into the drug composition. As a result, the adequate purging of oxygen from the composition's headspace facilitates the attainment of significantly more stable formulation of oxygen sensitive drug.

The present invention also includes a method of maintaining the stability of a pharmaceutical composition having hydrocortisone sodium phosphate as the active ingredient, including the slowing down or otherwise inhibiting of the oxidation pathway of hydrocortisone sodium phosphate by reducing the amount of oxygen in the process of manufacturing and/or storing of finished product. This can be done by replacing oxygen with nitrogen.

One embodiment of the invention relates to the filling of a stable, composition of hydrocortisone comprising the steps of flushing an empty container in the presence of an inert gas; maintaining an inert gas environment by filling an aqueous composition of hydrocortisone sodium phosphate into said container while consistently flushing with an inert gas; suitably minimizing the re-introduction of oxygen during steps and during transport of the container; vacuum treating the container headspace and purging the container headspace with an inert gas; repeating the vacuum treating and purging steps to control oxygen content in the container headspace; and sealing the container in a manner to minimize the presence of oxygen in the container headspace. The preferred container for this embodiment is a glass vial having a suitable opening for flushing with an inert gas and filling with a composition. It is preferred that the container is contemporaneously filled with the drug composition while purging with an inert gas. Nitrogen is the preferred inert gas.

The present invention uses dissolved oxygen in range of not more than or equal to about 1.0% oxygen to about not more than or equal to about 6.0% oxygen by volume of the headspace in the container.

In preferred embodiment, the present invention uses low oxygen levels of not more than or equal to 1.0% oxygen in the headspace of a container.

One preferred embodiment of the present invention relates to using low oxygen levels of not more than or equal to 2.0% oxygen in the headspace of a container.

In another preferred embodiment of the present invention low oxygen levels of not more than or equal to 4.0% oxygen in the headspace of a container is used.

Yet another preferred embodiment of the present invention relates to an aqueous pharmaceutical composition of hydrocortisone sodium phosphate with low oxygen levels of not more than or equal to 6.0% oxygen in the headspace of a container.

In an embodiment of present invention, the stability is determined with pH values, color index, monothioglycerol content, assay, and degradation profile of hydrocortisone sodium phosphate.

In preferred embodiment of present invention of pharmaceutical composition of hydrocortisone sodium phosphate further usings not more than 1.0% oxygen in headspace container is found to be stable.

In preferred embodiment of present invention, the aqueous pharmaceutical composition of hydrocortisone sodium phosphate remains stable for preferably at least 12 months, preferably at least 9 m or most preferably at least 6 months or further most preferably for 3 months at 25° C./60% RH (relative humidity) with low oxygen levels of not more than or equal to 1.0% oxygen in the headspace of a container.

According to another preferred embodiment of the invention, the composition of the invention remains stable for at least 12 months at 25° C./60% RH (relative humidity) with low oxygen levels of not more than or equal to 1.0% oxygen in the headspace of a container.

In another preferred embodiment of present invention, the aqueous pharmaceutical composition is stable with lower oxygen levels in the headspace correlated with the lower color value developed during storage.

As per yet of present invention, the aqueous pharmaceutical composition at 25° C., 12 months has a color value for the NMT 6% headspace oxygen level sample which is almost six times as high as that in samples with NMT 1% headspace oxygen.

The present invention is illustrated in more detail below in examples but is not intended to limit the scope of the present invention.

### Example 1: Representative Aqueous Pharmaceutical Composition of Hydrocortisone Sodium Phosphate. (Table 1)

| | | Quantity | Quantity | Quantity | Concentration | |
|---|---|---|---|---|---|---|
| Ingredients | Grades | (mg/2 mL) | (mg/ml) | (% w/v) | in milimole | Function |
| Hydrocortisone | USP | 134.2 | 67.1 | 6.71 | 137.95 | Active |
| sodium | | | | | | ingredient |
| phosphate | | | | | | |
| Dibasic sodium | USP | 4.4 | 2.2 | 0.22 | 15.49 | Buffering |
| phosphate | | | | | | agent |
| anhydrous | | | | | | |
| Monobasic | USP | 0.8 | 0.4 | 0.04 | 3.33 | |
| sodium | | | | | | |
| phosphate | | | | | | |
| anhydrous | | | | | | |
| Monothioglycerol | NF | 2.0 | 1.0 | 0.1 | 9.24 | Antioxidant |
| Sodium | NF | | q.s. to adjust the pH to 8.0 | | | pH adjusting |
| hydroxide | | | (Range: 7.5 to 8.5) | | | agent |
| Water for | USP | q.s. to | q.s. to | q.s. | NA | Vehicle |
| injection | | 2 mL | 1 mL | to 100% | | |

| | | | |
|---|---|---|---|
| Nitrogen | NF | q.s. | Processing |

aid

### Example 2: Process for Preparing the Composition of Hydrocortisone Sodium Phosphate Formulation

Procedure: An aqueous pharmaceutical composition was prepared by process comprising the following steps:

a) The water for injection was collected in jacketed SS compounding/manufacturing vessel and nitrogen was purged till the dissolved oxygen content was achieved.

b) Monothioglycerol, dibasic sodium phosphate anhydrous, monobasic sodium phosphate anhydrous and hydrocortisone sodium phosphate USP, were added under continuous stirring and nitrogen was purged in step (a) and the mixture was stirred till complete dissolution was achieved.

c) Hydrocortisone sodium phosphate USP acid was added under continuous stirring and nitrogen purging was carried out in step (b) and the solution was stirred till complete dissolution was achieved.

d) The pH of solution was adjusted to 8.0 by using NaOH solution and stirred.

e) The volume was made up using water to 100% of the batch volume and the mixture was stirred.

f) The mixture of step (e) was filtered and filled it into sterile container.

### Example 3: Stability Data of the Pharmaceutical Composition with Different Concentrations of Monothioglycerol

An aqueous pharmaceutical composition of hydrocortisone sodium phosphate is used to determine stability with different concentrations of monothioglycerol including about 0.5 mg/ml, about 1.0 mg/ml and about 2.0 mg/ml. The assay, color, monothioglycerol content and pH values of the samples were tested at time zero, 3 months, 6 months, 9 months, and 12 months at 25° C./60% RH (relative humidity). The assay performed by using HPLC, color index and pH was analyzed utilizing methodologies known or readily developed by those skilled in the art. The results are presented in table 2. As per data demonstrated in table 2, the assay, color index, pH value, monothioglycerol content and purity remain stable. Thus, monothioglycerol at about 1.0 mg/ml of was selected for further process.

TABLE 2
Representative results of stability data with different concentration
of monothioglycerol at 25° C./60% RH (relative humidity)

| | | | | | | Total |
|---|---|---|---|---|---|---|
| | | | | | | Degradation |
| | | | | | | products |
| | | | Colour | Monothioglycerol | | (Excluding |
| | | | index | content | Assay | Hydrocortisone |
| Test parameters | | pH | (AU) | (%) | (%) | impurity) |
| With 0.5 mg/ml of | Initial | 7.93 | NP | 81.7 | 103.3 | <0.1 |
| Monothioglycerol | 3 M | 7.88 | 0.0293 | 80.6 | 104.7 | NP |
| 25° C./60% RH | 6 M | 7.99 | 0.047 | 52.1 | 98.3 | <0.1 |
| | 9 M | 8 | 0.045 | 70.7 | 101.1 | <0.1 |
| | 12 M | 7.95 | 0.093 | NP | 103.1 | <0.1 |
| With 1.0 mg/ml of | Initial | 7.9 | NP | NP | 102.1 | <−0.1 |
| Monothioglycerol | 3 M | 7.84 | 0.047 | 38.7 | 98.1 | <−0.1 |
| 25° C./60% RH | 6 M | 7.84 | 0.1175 | 31.2 | 102.6 | <0.1 |
| | 9 M | 8.3 | 0.3576 | NP | NP | NP |
| | 12 M | 7.93 | 0.086 | 24.5 | 97.7 | <0.1 |
| With 2.0 mg/ml of | Initial | 8.04 | NP | 83.6 | 97.1 | <0.1 |
| Monothioglycerol | 3 M | 7.99 | 0.024 | 83.6 | 100.8 | <0.1 |
| 25° C./60% RH | 6 M | 8.23 | 0.022 | 82.1 | 98.4 | <0.1 |
| | 12 M | 8.15 | 0.062 | 73.4 | 97 | 0.14 |

### Example 4: Stability Data of the Pharmaceutical Composition with Different Headspace Oxygen (HSO) Level

Aqueous pharmaceutical compositions of hydrocortisone sodium phosphate with NMT 1% HSO, NMT 2% HSO, and NMT 6% HSO in vial were kept at 25° C./60% RH (relative humidity). The assay, color, monothioglycerol content and pH values of the sample were tested at time zero, 6 months, 9 months, and 12 months. Aqueous pharmaceutical composition of hydrocortisone sodium phosphate with NMT 4% HSO in vial was kept at 25° C./60% RH (relative humidity) and tested at time zero, 6 months, and 12 months.

TABLE 3
Representative results of stability data with different headspace
oxygen (HSO) level at 25° C./60% RH (relative humidity)

| | | | | | | Total |
|---|---|---|---|---|---|---|
| | | | | | | Degradation |
| | | | | | | products (%) |
| | | | Colour | Monothioglycerol | | (Excluding |
| | | | index | content | Assay | Hydrocortisone |
| Test parameters | | pH | (AU) | (%) | (%) | impurity) |
| (NMT 1% HSO in | Initial | 7.92 | 0.036 | 63.7 | 97.3 | <0.1 |
| Vial Headspace) | 6 M | 8 | 0.047 | 51.1 | 97.7 | 0.1 |
| 25° C./60% RH | 9 M | 7.95 | 0.054 | 60.6 | 98.9 | <0.1 |
| | 12 M | 8.12 | 0.0438 | 35.2 | 97.6 | 0.11 |
| | 18 M | 7.94 | 0.0547 | 23 | 97.1 | 0.16 |
| (NMT 2% HSO in | Initial | 7.89 | 0.0468 | 58.3 | 97.6 | <0.1 |
| Vial Headspace) | 6 M | 7.99 | 0.065 | 44.7 | 97.1 | 0.11 |
| 25° C./60% RH | 9 M | 7.84 | 0.072 | 38 | 99.6 | <0.1 |
| | 12 M | 8.02 | 0.0811 | 30.6 | 98.3 | 0.11 |
| | 18 M | 7.91 | 0.0669 | 19.7 | 97.3 | 0.26 |
| (NMT 4% HSO in | Initial | 7.89 | 0.134 | 38.4 | 97.4 | <0.1 |
| Vial Headspace) | 6 M | 7.94 | 0.137 | 26.7 | 95.8 | 0.13 |
| 25° C./60% RH | 12 M | 8.1 | 0.0682 | 12.1 | 98.4 | <0.1 |
| (NMT 6% HSO in | Initial | 7.92 | 0.2281 | 8.6 | 97.3 | <0.1 |
| Vial Headspace) | 6 M | 7.99 | 0.178 | 0.2 | 95 | 0.13 |
| 25° C./60% RH | 9 M | 7.95 | 0.203 | 67.3 | 98.5 | 0.13 |
| | 12 M | 8.03 | 0.2834 | ND | 98.1 | 0.41 |
| | 18 M | 7.89 | 0.3147 | ND | 98 | 0.14 |

The assay was performed using HPLC method. The color index was generated utilizing methodologies known or readily developed by those skilled in the art. The results are presented in table 3. The pH of the composition was found in between 7.5 to 8.5. According to the invention, there was no significant impact of headspace on assay and degradation profile of hydrocortisone sodium phosphate but there was increase in color index with increase in oxygen level in the headspace in vials. As per data in table 3 the aqueous pharmaceutical composition of hydrocortisone sodium phosphate remains stable with headspace oxygen levels of NMT 2%. Lower oxygen levels in the headspace correlated with the lower color value developed during storage. At 25° C. and 12 months, the color value for the NMT 6% headspace oxygen level sample was almost six times as high as that in samples with NMT 1% headspace oxygen.

1. A safe, ready-to-use aqueous pharmaceutical composition comprising:
hydrocortisone sodium phosphate or a pharmaceutically acceptable salt thereof, and a pharmaceutically acceptable excipient.

2. The composition according to claim 1, wherein the pharmaceutically acceptable excipients are selected from an antioxidant, a buffering agent, a pH adjuster and a vehicle.

3. The composition according to claim 1, wherein hydrocortisone sodium phosphate is present in a concentration of about 0.1% w/v to about 100% w/v.

4. The composition according to claim 3, wherein hydrocortisone sodium phosphate is present in a concentration of about 1% w/v to about 10% w/v.

5. The composition according to claim 4, wherein hydrocortisone sodium phosphate is present in a concentration of an amount of about 6.71% w/v.

6. The composition according to claim 2, wherein the buffering agents are present in a concentration of about 0.01% w/v to about 0.5% w/v.

7. The composition according to claim 6, wherein the buffering agents are present in a concentration of about 0.02% w/v to about 0.3% w/v.

8. The composition according to claim 2, wherein the buffering agents are selected from dibasic sodium phosphate anhydrous, monobasic sodium phosphate anhydrous or mixtures thereof.

9. The composition according to claim 8, wherein dibasic sodium phosphate anhydrous is present in a concentration of about 0.01% w/v to about 0.5% w/v.

10. The composition according to claim 9, wherein dibasic sodium phosphate anhydrous is present in a concentration of about 0.02% w/v to about 0.3% w/v.

11. The composition according to claim 10, wherein, dibasic sodium phosphate anhydrous is present in a concentration of about 0.22% w/v.

12. The composition according to claim 8, wherein monobasic sodium phosphate anhydrous is present in a concentration of about 0.02% w/v to about 0.3% w/v.

13. The composition according to claim 12, wherein monobasic sodium phosphate anhydrous is present in a concentration of about 0.04% w/v.

14. The composition according to claim 2, wherein the pH adjuster is selected from sodium hydroxide, sodium borate and sodium carbonate.

15. The composition according to claim 14, wherein the pH adjuster is sodium hydroxide.

16. The composition according to claim 15, wherein the sodium hydroxide is used to adjust the pH to a range of about 7.5 to about 8.5.

17. The composition according to claim 16, wherein the sodium hydroxide is used to adjust the pH to about 8.0.

18. The composition according to claim 2, wherein the antioxidant is present in a concentration of about 0.005% w/v to about 1.0% w/v.

19. The composition according to claim 18, wherein the antioxidant is present in a concentration range of about 0.05% w/v to about 5.0% w/v.

20. The composition according to claim 2, wherein the antioxidant is monothioglycerol.

21. The composition according to claim 20, wherein monothioglycerol is present in a concentration of about 0.01% w/v to about 0.5% w/v.

22. The composition according to claim 21, wherein monothioglycerol is present in a concentration of about 0.05% w/v to about 0.25% w/v.

23. The composition according to claim 22, wherein monothioglycerol is present in a concentration of about 0.1% w/v.

24. The composition according to claim 2, wherein the vehicle is water for injection.

25. The composition according to claim 20, wherein the concentration of monothioglycerol is selected from about 0.5 mg/ml, about 1.0 mg/ml or about 2.0 mg/ml.

26. The composition according to claim 1, wherein the said composition does not contain sulfite.

27. The composition according to claim 1, wherein the said composition is administered by parenteral, intramuscular, or subcutaneous route.

28. A safe, ready-to-use aqueous pharmaceutical composition comprising: a) hydrocortisone sodium phosphate in an amount of about 6.71% w/v; b) a mixture of dibasic sodium phosphate in an amount of about 0.22% w/v and monobasic sodium phosphate in an amount of about 0.04% w/v; and c) monothioglycerol in an amount of about 0.1% w/v.

29. The composition according to claim 1, wherein the said composition is prepared by a process comprising the following steps:
a) collecting a sufficient quantity of a required vehicle in a manufacturing vessel and flushing inert gas into it until a dissolved oxygen content is achieved;
b) maintaining an inert gas condition and suitably adding an antioxidant and buffering agent into step a), stirring the solution until complete dissolution;
c) adding active ingredients into step b) by maintaining a suitable condition until complete dissolution;
d) adjusting the pH of the solution with the help of a pH adjuster;
e) making up a volume with a vehicle; and
f) filtering it and filling it into sterilized containers.

30. The composition according to claim 29, wherein the said composition is prepared by a process comprising the following steps:
a) collecting water for injection in jacketed SS compounding/manufacturing vessel and purging nitrogen until the dissolved oxygen content is achieved;
b) adding monothioglycerol, dibasic sodium phosphate anhydrous, monobasic sodium phosphate anhydrous and hydrocortisone sodium phosphate USP, under continuous stirring;
c) adding hydrocortisone sodium phosphate USP acid under continuous stirring and nitrogen purging in step (b) and stirring the solution until complete dissolution;
d) checking and adjusting the pH of the solution to 8.0;
e) making up the volume with water for injection to 100% of a batches volume and stirring; and
f) filtering the solution of step (e) and filling it into sterilized containers.
